(19) United States
(12) Patent Application Publication
(10) Pub. No.: US 2021/0290450 A1
(43) Pub. Date: Sep. 23, 2021

(54) THREE-DIMENSIONAL FOAM-LAID NONWOVENS

(71) Applicant: KIMBERLY-CLARK WORLDWIDE, INC., Neenah, WI (US)

(72) Inventors: Davis Dang H. Nhan, Menasha, WI (US); Cathleen M. Uttecht, Menasha, WI (US); Lori A. Eslinger, Appleton, WI (US); Neil T. Scholl, Neenah, WI (US); Jian Qin, Appleton, WI (US); Charles W. Colman, Marietta, WI (US); Deborah J. Calewarts, Winneconne, WI (US); Vasily A. Topolkaraev, Appleton, WI (US); Antonio J. Carrillo Ojeda, Appleton, WI (US)

(73) Assignee: KIMBERLY-CLARK WORLDWIDE, INC., Neenah, WI (US)

(21) Appl. No.: 17/262,378

(22) PCT Filed: Jul. 25, 2018
(86) PCT No.: PCT/US18/43657

§ 371 Date: Jan. 22, 2021

(51) Int. Cl. A61F 13/511; A61F 13/494; A61F 13/513

(52) U.S. Cl. CPC A61F 13/51121; A61F 13/494; A61F 2013/51134; A61F 13/513; A61F 13/51104

(57) ABSTRACT

A high topography nonwoven substrate includes synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, and wherein the density of a projection element is the same as the density of the base layer.

### BACKGROUND

A bowel movement (BM) leaking from a diaper (i.e., leak around the leg area or at the waist) causes an unpleasant mess needing clean up by a caregiver. The consumer/chooser becomes dissatisfied with the absorbent product of choice, which can lead to the consumer/chooser deciding to switch to different diaper brand. As many as one in seven diapers containing BM result in BM leaking from the diaper. In addition, BM that is in contact with skin can result in compromising skin health and promoting development of diaper rash. Non-diaper skin can be healthier than diapered skin because current diapers do a poor job of keeping BM off of skin.

There is a lack of material/nonwoven solutions to reduce BM leakage occurrences and keep BM off of skin. Current materials in absorbent products, such as spunbond, SMS, and BCW, are mostly flat, dense, and do a poor job of handling runny BM and keeping BM off of skin. There are materials such as aperture films and textured BCW/SB composite nonwovens (e.g., TEXTOR brand nonwovens) that are used as liners. TEXTOR brand nonwovens can improve BM management properties compared to spunbond liner and is used in current products. Too many current products, however, that contain BM result in BM leakage. As a result, there is a great opportunity to identify materials that improve absorbent product BM management performance.

### SUMMARY

The materials of the present disclosure are the next step in producing a diaper that completely absorbs runny BM at the point of insult, leaving no BM spreading and no BM on the skin, to deliver zero BM leakage and a cleaner skin experience. Identifying solutions to reduce BM blow outs and BM on skin is beneficial both to the wearer of the product and because it would result in a consumers having a more positive experience with such products by reducing the occurrence of diaper rash and providing a point of differentiation from other products.

The solutions disclosed herein are nonwoven materials having high degrees of three-dimensional (3D) topography and that have high compression resistance while also having a high level of openness. Such materials have demonstrated significantly better BM intake compared to current commercial materials being using in current products. The BM Flat Plate test method has demonstrated that three-dimensional foam-laid webs of the present disclosure reduce BM pooling to 2% wt/wt versus TEXTOR brand nonwovens at 40% wt/wt. BM pooling values are similar to rewet values and represent BM on skin.

The present disclosure describes novel extreme 3D nonwoven materials that have superior BM management properties. Such materials can improve absorbent products by reducing BM leakage and BM on skin. The nonwoven structures are made possible by templating foam-laid webs, otherwise labeled as 3D foam-laid nonwovens. The process involves dispersing bicomponent fibers in foam and templating such foam during drying & thermal bonding. This method results in extreme 3D nonwoven webs with features as high as 12 mm in height and as low as 8 mm in diameter. Because of these 3D features, there is high level of Z-direction fiber orientation that results in webs having high compression resistance while also having a high level of openness/porosity, which are key properties in being able to handle runny BM. In addition, a wide variety of 3D features, shapes, and sizes can be produced depending on template design.

The present disclosure is generally directed to a high topography nonwoven substrate including synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, and wherein the density of a projection element is the same as the density of the base layer.

In another aspect, the present disclosure is generally directed to a high topography nonwoven substrate including synthetic binder fibers, wherein the fibers of the substrate are entirely synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, wherein the shape of a cross-section of a projection element at the proximal end of the projection element is the same as the shape of a cross-section of a projection element at the distal end of the projection element, and wherein the density of a projection element is the same as the density of the base layer

In still another aspect, the present disclosure is generally directed to a high topography nonwoven substrate includes synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, wherein each projection element has a uniform density, wherein the height of a projection element is greater than the width or diameter of that projection element, and wherein the density of a projection element is the same as the density of the base layer.

Various features and aspects of the present disclosure will be made apparent from the following detailed description.

### BRIEF DESCRIPTION OF THE FIGURES

A full and enabling disclosure of the present disclosure, including the best mode thereof to one of ordinary skill in the art, is set forth more particularly in the specification, including reference to the accompanying figures in which:

FIG. 1 is a flowchart view of an exemplary aspect of a process for producing 3D foam-laid nonwovens in accordance with the present disclosure;

FIG. 2 is a perspective schematic illustration of one aspect of a template for use in the process of FIG. 1;

FIG. 3 photographically illustrates the results of Flow Through testing of various nonwovens including those produced by the process of FIG. 1;

FIG. 4 graphically illustrates the results of Flow Through testing of various nonwovens including those produced by the process of FIG. 1;

FIG. 5 graphically illustrates the results of Compression Resistance testing of various nonwovens including those produced by the process of FIG. 1; and

FIG. 6 graphically illustrates the results of Air Permeability testing of various nonwovens including those produced by the process of FIG. 1.

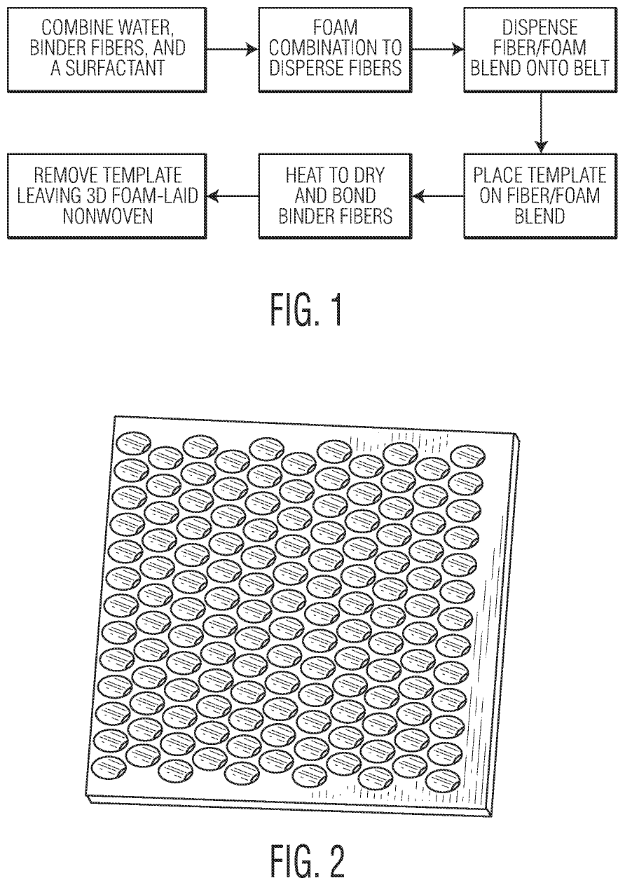

Repeated use of reference characters in the present specification and drawings is intended to represent the same or analogous features or elements of the present disclosure.

### DETAILED DESCRIPTION

Reference now will be made to the aspects of the disclosure, one or more examples of which are set forth below. Each example is provided by way of explanation of the disclosure, not as a limitation of the disclosure. In fact, it will be apparent to those skilled in the art that various modifications and variations can be made in the disclosure without departing from the scope or spirit of the disclosure. For instance, features illustrated or described as part of one aspect can be used on another aspect to yield a still further aspect. Thus, it is intended that the present disclosure cover such modifications and variations as come within the scope of the appended claims and their equivalents. It is to be understood by one of ordinary skill in the art that the present discussion is a description of exemplary aspects only, and is not intended as limiting the broader aspects of the present disclosure, which broader aspects are embodied in the exemplary constructions.

The present disclosure describes novel extreme 3D nonwoven materials that have superior BM management properties. Such materials can improve absorbent products by reducing BM leakage and BM on skin. The nonwoven structures are made possible by templating foam-laid webs, otherwise labeled as 3D foam-laid webs. The process involves dispersing bicomponent fibers in foam and templating such foam during drying & thermal bonding. This method results in extreme 3D nonwoven webs with relatively tall and narrow 3D features. Because of these 3D features, there is high level of Z-direction fiber orientation that results in webs having high compression resistance while also having a high level of openness/porosity, which are key properties in being able to handle runny BM. In addition, a wide variety of 3D features, shapes, and sizes can be produced depending on template design.

Foam processes are normally used for making planar webs having a uniform thickness, such as two-dimensional shaped forms. As described herein, a three-dimensional nonwoven web is created by using a three-dimensional template to mold foam into a 3D topography. Drying and heating the templated foam results in a nonwoven having the topographical characteristics of the template.

The process of the present disclosure obviates any further molding of the nonwoven web as any desired topography is created along with the creation of the nonwoven. Prior methods of processing nonwovens require post-creation manipulation, cutting, embossing, or molding of an existing nonwoven web, resulting in weakening the web along with broad variations in web density and basis weight.

Creation of the nonwoven structures described herein requires three major steps: 1) Disperse binder fiber and a foaming agent in water to create a foam solution with a consistency some describe as shaving cream-like. 2) Template the fiber/foam blend. 3) Dry and heat the blend to remove water and to activate the binder fibers, thus setting the 3D structure in the nonwoven. These webs are referred to herein as 3D foam-laid nonwovens.

In the first step, binder fiber and a foaming agent are dispersed in water to create a foam solution with a consistency some describe as shaving cream-like. This step involves dispersing a blend of fibers capable of forming fiber-fiber bonds (e.g., bicomponent fibers/binder fibers) in a foam solution. This is done by mixing fibers, water, and foaming agent such as sodium dodecyl sulfate (SDS) surfactant simultaneously to create a foam and to uniformly suspend the fibers in the foam. This foaming process creates a stable foam containing a fibrous network that is uniformly dispersed through the foam solution. The foam has high viscosity, preventing fibers from floating, sinking, and/or agglomerating.

Many types of fibers can be included in the fiber blend, but the blend must include binder fibers in a quantity sufficient to ensure the final 3D foam-laid nonwoven has integrity and can maintain its 3D structural features. In one example, the fiber blend is 100% wt/wt binder fibers having a polyethylene sheath and a polypropylene core. The binder fibers are typically synthetic, thermoplastic bonder fibers. In other aspects, the binder fibers can be bi- and/or multi-component binder fibers. In other aspects, the fiber blend can include cellulosic fibers.

In another aspect of the present disclosure, nanovoided technology has produced lightweight, uncrimped bicomponent staple fiber with a fiber density reduction of 20-33%. Use of such lightweight fibers in the fiber blend can increase the fiber count for the same basis weight, thus increasing the web compression resistance. In various aspects, the low-density fiber can have density as low as 0.5 grams/cubic centimeter or even lower. In an example, the low density-voided fibers used can have a density of 0.62 g/cc, which equates to the polyolefin-based fiber having a 33% reduction in overall density with a 47% void volume in the core. Foam forming is a preferred method of forming a nonwoven web containing low-density fibers and enables lofty webs using voided fibers that do not require stuffer box crimped fibers. For example, carded webs require fibers to be stuffer box crimped to form a web. Stuff box crimping is a high pressure process that results in internal fiber void structure destruction and thus cannot generate a carded web including low-density voided fibers. Because of the high viscosity foam, low-density fibers are able to be properly laid into a web using the foam as carrier, thus enabling the ability to form webs including low-density fibers.

Although some level of binder fibers is required, the fiber blend does not need to contain solely binder fibers; other types of fibers can be incorporated into the fiber blend. The selection of fibers can include all types of synthetic fibers to a wide range of natural fibers. The fibers can have a wide range of cut length/fiber length, such as from 3-30 mm. A wide range of fiber diameters can also be used. A wide variety of foaming agents and amounts can be used such as anionic and non-ionic in amounts ranging from 0.1- 5wt%. Typically,

SDS has been used at about 0.17 wt % to water. Foam density can range from 100-400 g/L. Foam stability half-life can range from 2-30 min. Fiber consistency (fiber concentration) can range from 0.5-5% wt/wt.

In the second step, the fiber/foam blend is poured onto or applied in any suitable manner to a foraminous belt or other suitable surface. The belt optionally includes a frame-type mold to limit the spread of the fiber/foam blend on the belt. A template is then placed on top of the fiber/foam blend, typically within the mold if present. The template provides a negative pattern to the desired pattern of the 3D foam-laid nonwoven. In one illustrative example, if a convex surface is desired for the nonwoven, then the template will have a concave surface pattern. Upon placement of the template, the fiber/foam blend conforms to the topography of the template, in essence creating bumps of foam where the template has dimples, dimples where the template has bumps, and flat spaces where the template is flat. In this manner, the template creates a 3D topography in the foam.

Typically the template contains cavities into which the fiber/foam blend can flow and fill. Cavity sizes range from 8 mm in diameter or larger and cavity depths can be as large as the thickness of the applied foam, as large as 50 mm or more. In one example, the template cavities are 12 mm deep. The cavities can have any suitable shape, including round, rectangular, square, triangular, mushroom-shaped, symbols, toroidal, or more complex combinations of shapes, and the template cavities can have any combination of shapes, sizes, and depths, or the template cavities can be of one uniform shape, size, and depth, as long as the fiber/foam blend can flow and fill the cavities in the template.

The template material should be selected to withstand bonding temperatures. Examples of template materials include silicon, metal, polyurethane, polytetrafluoroethylene, and any other suitable material. The template material should also be selected such that fibers do not adhere to template, thereby allowing easy removal of the web from the template, or the template from the web, after thermal activation of the binder fiber. In other words, the binder fibers should preferably adhere to other binder fibers rather than the template material. In general, increased fiber-to-fiber bonding overcomes fiber-to-template bonding problems. The template should also be open enough to allow proper air flow and heat transfer to enable drying and heat activation of the binder fiber.

In the third step, the templated fiber/foam blend is placed in an oven or other suitable heating device to dry and thermally bond the binder fibers. It is important that the template is present during the drying/bonding stage to ensure the 3D structure will be present in the final web. Temperatures and time in the oven should be long enough to remove a sufficient amount of water and to sufficiently activate the binding fiber. The time and temperature can be set by one skilled in the art based on the ingredients in the fiber/foam blend, the volume and surface area of the fiber/foam blend, the specifications of the oven used, the initial conditions of the templated fiber/foam blend, and any other relevant conditions.

The process described herein produces unique webs. Different high topography 3D nonwovens can be produced by selecting different templates (e.g., templates with different cavity sizes, shapes, depths, spacing, etc.). The 3D foam-laid nonwovens produced by the process described herein typically have a base layer defining an X-Y plane, where the base layer has an X-Y surface and a backside surface opposite the X-Y surface.

The 3D foam-laid nonwovens also include vertical (Z-direction) features such as projection elements protruding in the Z-direction from and integral with the base layer. This is often called a “peak and valley” type 3D structure. Each projection element has a height, a diameter or width, a cross-section, a sidewall, a proximal end where the projection element meets the base layer, and a distal end opposite the proximal end. The projection elements are typically distributed in both the X- and Y-directions. The projection elements can be uniformly distributed in both the X- and Y-directions, or the pattern of projection elements can be varied in either or both directions.

Depending on the template design, may different vertical feature shapes and sizes can be created. For example, a horizontal cross-section of a projection element can have any suitable shape, including round, rectangular, square, triangular, mushroom-shaped, symbols, toroidal, or more complex combinations of shapes. The height of the vertical features can range from 1 mm to 50 mm or greater, 1 mm to 30 mm, 5 mm to 50 mm, 5 mm to 30 mm, 30 mm to 50 mm, or any other suitable range of heights. The width or diameter of a vertical feature, depending on the shape of its cross-section, can be 8 mm or greater. The heights of the projection elements are preferably greater than the width or diameter of the projection elements. In various aspects, the ratio of the height of a projection element to the width or diameter of that projection element is greater than 0.5.

Because of the manner in which the 3D foam-laid nonwoven is produced, the density of a projection element is generally the same as or similar to the density of the base layer. In various aspects, the shape of a cross-section of a projection element at the proximal end of the projection element is the same as the shape of a cross-section of a projection element at the distal end of the projection element. Alternatively, the shape of a cross-section of a projection element at the proximal end of the projection element can be different from the shape of a cross-section of a projection element at the distal end of the projection element. The density of a projection element at the proximal end of the projection element can be the same as or different from the density of a projection element at the distal end of the projection element. The basis weight of a projection element at the proximal end of the projection element can be the same as or different from the density a projection element at the distal end of the projection element. In other aspects, the size of a cross-section of a projection element at the proximal end of the projection element can be the same as or different from the size of a cross-section of a projection element at the distal end of the projection element.

Each projection element can have an internally-uniform density. In other words, each projection element generally has a homogeneous density largely free of hollow or densified portions. The projection elements can have a density between 0.001 and 0.02 g/cc. The 3D foam-laid nonwovens demonstrate a basis weight range from 15 gsm to 120 gsm, although lower or higher basis weights can be produced using the process described herein.

Because of the manner in which the 3D foam-laid nonwoven is produced, the projection elements and particularly the sidewalls of the projection elements have fibers aligned in the Z-direction. In some aspects, the sidewalls have greater than 50 percent of fibers oriented in the Z-direction. Because of high degree of fiber z-direction orientation, the 3D foam-laid nonwovens described herein demonstrate very high compression resistance while also being very open and having a high level of porosity. For the purpose of comparison, “flat” bonded carded web (BCW) surge provides compression resistance of about 25 cc/g at 0.6 kPa pressure. The 3D foam-laid nonwovens of the present disclosure provide compression resistance from about 35 up to 65 cc/g at 0.6 kPa pressure. In addition, these high level of compression resistance is achievable with very open web structures.

Again for the purpose of comparison, 100 gsm MGL9 surge, a standard BCW-type surge material, has an air permeability value of about 440 cfm, while the 3D foam-laid nonwovens measure between 1000 and 2500 cfm.

Bench testing of 3D foam-laid nonwovens has demonstrated superior BM management properties. For example, a test method for BM flow measures the amount of BM simulant that transfers from a BM-simulant-insulted nonwoven to blotter paper. A liner made from TEXTOR brand nonwoven typically results in about 40% of BM simulant remaining on the surface of the liner as shown using the blotter paper (i.e., the amount remaining is also called % pooled). The 3D foam-laid nonwovens of the present disclosure have demonstrated about half the amount of % pooling (i.e., 20%) compared to the TEXTOR brand nonwoven at about half the basis weight of TEXTOR brand nonwoven (55 gsm TEXTOR brand nonwoven compared to 30 gsm 3D foam-laid nonwoven). At higher basis weights, such as a 60 gsm 3D foam-laid nonwoven, demonstrated less than 2% BM simulant % pooled. The % pooled indicator can be consider analogous to “what is on skin” or rewet.

### EXAMPLES

### Procedures

### Air Permeability Test

Air Permeability was measured in cubic feet of air per minute passing through a 38 square cm area (circle with 7 cm diameter) using a Textest FX3300 air permeability tester manufactured by Textest Ltd., Zurich, Switzerland. All tests were conducted in a laboratory with a temperature of 23±2° C. and 50±5% relative humidity. Specifically, a nonwoven sheet is allowed to dry out and condition for at least 12 hours in the 23±2° C. and 50±5% relative humidity laboratory before testing. The nonwoven sheet is clamped in the 7 cm diameter sheet test opening and the tester is set to a pressure drop of 125 Pa. Placing folds or crimps above the fabric test opening is to be avoided if at all possible. The unit is turned on by applying clamping pressure to the sample. The air flow under the 125 Pa pressure drop is recorded after 15 seconds of airflow to achieve a steady state value.

The Air Permeability Test measures the rate of airflow through a known dry specimen area. The air permeability of each sample was measured using a Textest FX3300 air permeability tester available from Schmid Corporation, having offices in Spartanburg, S.C.

A specimen from each test sample was cut and placed so the specimen extended beyond the clamping area of the air permeability tester. The test specimens were obtained from areas of the sample that were free of folds, crimp lines, perforations, wrinkles, and/or any distortions that make them abnormal from the rest of the test material.

The tests were conducted in a standard laboratory atmosphere of 23±1° C. (73.4±1.8° F.) and 50±2% relative humidity. The instrument was turned on and allowed to warm up for at least 5 minutes before testing any specimens. The instrument was calibrated based on the manufacturer's guidelines before the test material was analyzed. The pressure sensors of the instrument were reset to zero by pressing the NULL RESET button on the instrument. Before testing, and if necessary between samples or specimens, the dust filter screen was cleaned, following the manufacturer's instructions. The following specifications were selected for data collection: (a) Unit of measure: cubic feet per minute (cfm); (b) test pressure: 125 Pascal (water column 0.5 inch or 12.7 mm); and (c) test head: 38 square centimeters (cm²). Because test results obtained with different size test heads are not always comparable, samples to be compared should be tested with the same size test head.

The NULL RESET button was pressed prior to every series of tests, or when the red light on the instrument was displayed. The test head was open (no specimen in place) and the vacuum pump was at a complete stop before the NULL RESET button was pressed.

Each specimen was placed over the lower test head of the instrument. The test was started by manually pressing down on the clamping lever until the vacuum pump automatically started. The Range Indicator light on the instrument was stabilized in the green or yellow area using the RANGE knob. After the digital display was stabilized, the air permeability of the specimen was displayed, and the value was recorded. The test procedure was repeated for 10 specimens of each sample, and the average value for each sample was recorded as the air permeability.

### Compression Test Method

From the target nonwoven, a 38 mm by 25 mm test sample was cut. The upper and lower platens made of stainless steel were attached to a tensile tester (Model: Alliance RT/1 manufactured by MTS System Corporation, a business having a location in Eden Prairie, Minn., U.S.A.). The top platen had a diameter of 57 mm while the lower platen had a diameter of 89 mm. The upper platen was connected to a 100 N load cell while the lower platen was attached to the base of the tensile tester. TestWorks Version 4 software program provided by MTS was used to control the movement of the upper platen and record the load and the distance between the two platens. The upper platen was activated to slowly move downward and touch the lower platen until the compression load reached around 5000 g. At this point, the distance between the two platens was zero. The upper platen was then set to move upward (away from the lower platen) until the distance between the two platens reaches 15 mm. The crosshead reading shown on TestWorks Version 4 software program was set to zero. A test sample was placed on the center of the lower platen with the projections facing toward the upper platen. The upper platen was activated to descend toward the lower platen and compress the test sample at a speed of 25 mm/min. The distance that the upper platen travels was indicated by the crosshead reading. This was a loading process. When 345 grams of force (about 3.5 kPa) was reached, the upper platen stopped moving downward and returned at a speed of 25 mm/min to its initial position where the distance between the two platens was 15 mm. This was an unloading process. The compression load and the corresponding distance between the two platens during the loading and unloading were recorded on a computer using TestWorks Version 4 software program provided by MTS. The compression load was converted to the compression stress by dividing the compression force by the area of the test sample. The distance between the two platens at a given compression stress represented the thickness under that particular compression stress. A total of three test samples were tested for each test sample code to get representative loading and unloading curves for each test sample code.

### Flow Through Test Method

The Flow Through Test was performed using Simulant A, which was applied to the targeted nonwoven. The BM simulant was applied using a BM gun and the absorption test conducted using the BM Plate Test Method. The targeted nonwoven was the material described herein. The four corners of the BM plate were then adjusted to match nonwoven thickness and checked to make sure the plate was level. The nonwoven was placed between the lower and upper plates and insulted with BM simulant. The nonwoven was left in the test apparatus for 2 minutes after insult, and then placed on a vacuum box to measure the amount of BM simulant pooling on the nonwoven. Four paper towels were placed on top of the nonwoven and the nonwoven was flipped with the paper towels down on top of the vacuum box and covered with a silicone sheet to seal the vacuum. The vacuum box was turned on pulling a pressure of 5 inches of water for 1 minute. In addition to the BM simulant picked-up by paper towels on the vacuum box, the excess BM simulant left on the BM plate was removed using an additional paper towel. The BM simulant amount picked up by the paper towels from the vacuum box along with the excess BM simulant left on the plate was recorded as the total pooled BM simulant.

Three (N=3) samples were tested for each of the examples. The amounts of BM simulant in each layer in the 3 samples were then averaged to get BM simulant pooled on the nonwoven.

### Materials

### Fibers

Voided bicomponent fibers with a diameter of 33 microns, a denier of 5.5 dpf, and a density of 0.705 g/cc. Non-voided bicomponent fibers with a diameter of 33 microns, a denier of 7.1 dpf, and a density of 0.913 g/cc. Note that the density of the voided bicomponent fibers was 23 percent less than the density of the non-voided bicomponent fibers. The fiber density was measured using sink/float after web thermal bonding at 133° C. The fibers were cut to a length of 18 mm, then heat set at 118° C. for a final length of 15 mm. The codes tested are listed in Table 1.

TABLE 1
Codes Tested

| Code | | Target Basis |
|---|---|---|
| Number | Fiber | Weight, in gsm |
| 1 | Voided | 30 |
| 2 | Voided | 60 |
| 3 | Voided | 120 |
| 4 | Non-Voided | 30 |
| 5 | Non-Voided | 60 |
| 6 | Non-Voided | 120 |

The three-dimensional foam-laid handsheets tested herein were produced by combining 300 grams of deionized water, 5 grams of 10% SDS, and fibers. The combination was mixed to foam and poured into an 8 inch by 8 inch by 2 inch frame. This was then templated with a template having square holes of 1 cm, an openness of 40%, and a thickness of 12 mm, with a nylon spunbond backing. The assembly was dried and thermal bonded at 133° C. for 1 to 1.5 hours. This was then wet dipped in 0.2% wt/wt of SILWET brand DA63 surfactant in water and dried in ambient conditions.

### Fecal Material Simulant

The following is a description of the fecal material simulant A used in the examples described herein.

Ingredients:
DANNON brand All Natural Low-fat Yogurt (1.5% milkfat grade A), Vanilla with other natural flavor, in 32 oz container.
MCCORMICK brand Ground Turmeric
GREAT VALUE brand 100% liquid egg whites
KNOX brand Original Gelatin—unflavored and in powder form
DAWN brand Ultra Concentrated original scent dishwashing liquid
Distilled Water
Note: All fecal material simulant ingredients can be purchased at grocery stores such as WAL-MART brand stores or through on-line retailers. Some of the fecal material simulant ingredients are perishable food items and should be incorporated into the fecal material simulant at least two weeks prior to their expiration date.

### Mixing Equipment:

Laboratory Scale with an accuracy to 0.01 gram
500 mL beaker
Small lab spatula
Stop watch
IKA-WERKE brand Eurostar Power Control-Vise with R 1312 Turbine stirrer available from IKA Works, Inc., Wilmington, N.C., USA.

### Mixing Procedure:

1. A 4-part mixture is created at room temperature by adding, in the following order, the following fecal material simulant ingredients (which are at room temperature) to the beaker at a temperature between 21° C. and 25° C.: 57% yogurt, 3% turmeric, 39.6% egg white, and 0.4% gelatin. For example, for a total mixture weight of 200.0 g, the mixture will have 114.0 g of the yogurt, 6.0 g of the turmeric, 79.2 g of the egg whites, and 0.8 g of the gelatin.

2. The 4-part mixture should be stirred to homogeneity using the IKA-WERKE brand Eurostar stirrer set to a speed of 50 RPM. Homogeneity will be reached in approximately 5 minutes (as measured using the stop watch). The beaker position can be adjusted during stirring so the entire mixture is stirred uniformly. If any of the mixture material clings to the inside wall of the beaker, the small spatula is used to scrape the mixture material off the inside wall and place it into the center part of the beaker.

3. A 1.3% solution of DAWN brand dishwashing liquid is made by adding 1.3 grams of DAWN brand Ultra Concentrated dishwashing liquid into 98.7 grams of distilled water. The IKA-WERKE brand Eurostar and the R 1312 Turbine stirrer is used to mix the solution for 5 minutes at a speed of 50 RPM.

4. An amount of 2.0 grams of the 1.3% DAWN brand dishwashing liquid solution is added to 200 grams of the 4-part mixture obtained from Step 2 for a total combined weight of 202 grams of fecal material simulant. The 2.0 grams of the 1.3% DAWN brand dishwashing liquid solution is stirred into the homogenous 4-part mixture carefully and only to homogeneity (approximately 2 minutes) at a speed of 50 RPM, using the IKA-WERKE brand Eurostar stirrer. Final viscosity of the final fecal material simulant should be 390±40 cP (centipoise) when measured at a shear rate of 10 s⁻¹ and a temperature of 37° C.

5. The fecal material simulant is allowed to equilibrate for about 24 hours in a refrigerator at a temperature of 7° C. It can be stored in a lidded and airtight container and refrigerated for up to 5 days at around 7° C. Before use, the fecal material simulant should be brought to equilibrium with room temperature. It should be noted that multiple batches of fecal material simulant of similar viscosity can be combined. For example, five batches of fecal material simulant of similar viscosity and each 200 grams can be combined into one common container for a total volume of 1000 cc. It will take approximately 1 hour for 1000 cc of fecal material simulant to equilibrate with room temperature.

### Results

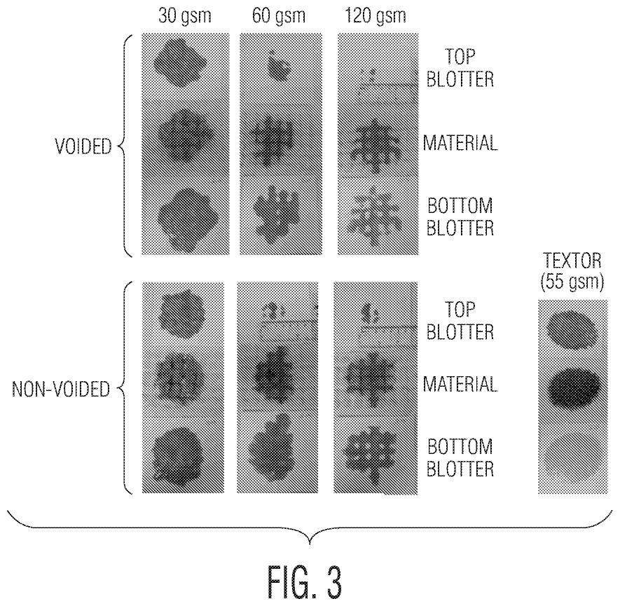

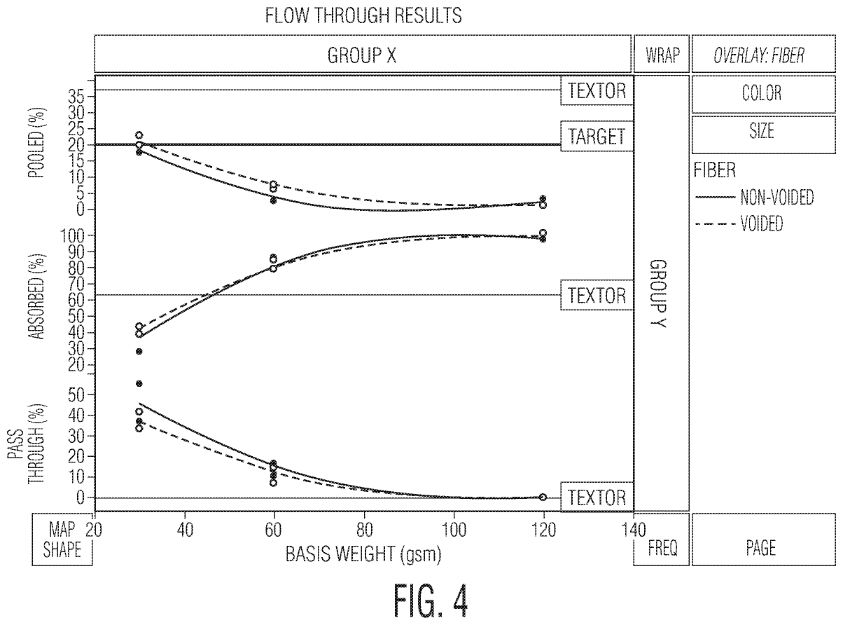

Results of the Flow Through Tests are illustrated in FIGS. 3 and 4. 3D foam-laid nonwovens using voided and non-voided binder fibers behaved similarly in the tests. When compared to the results of the TEXTOR brand nonwoven test, the 3D foam-laid nonwoven test demonstrated approximately half the % pooling at nearly half the basis weight. Higher basis weight 3D foam-laid nonwovens demonstrated approximately less than 2% % pooling. In addition, there is a higher level of BM simulant passing through the 3D foam-laid nonwoven, even at a basis weight of 60 gsm.

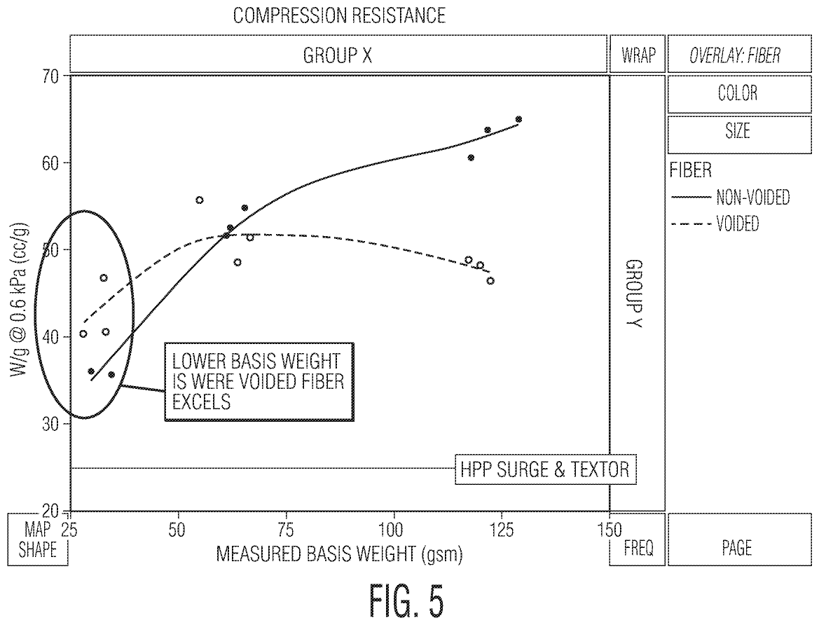

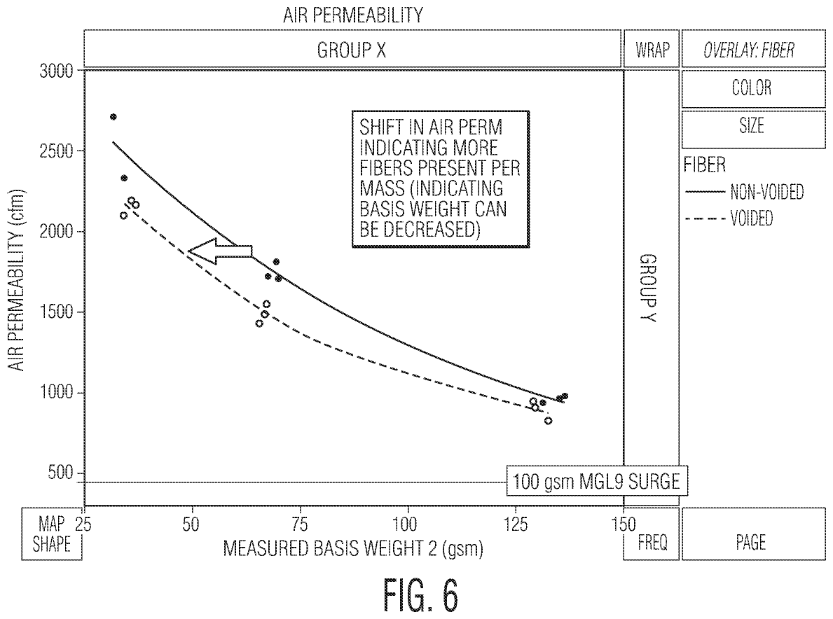

Results of compression and air permeability tests are illustrated in FIGS. 5 and 6. The 3D foam-laid nonwovens of the present disclosure exhibit high compression resistance and high air permeability. In this peak and valley model, the combination of open valleys and compression resistant peaks is an arrangement resulting in low % pooling values.

The solutions disclosed herein are nonwoven materials having high degrees of 3D topography, high compression resistance, and a high level of openness. Such materials demonstrate significantly better BM intake compared to current commercial materials used in current products. The BM Flat Plate test method has demonstrated that the 3D foam-laid nonwovens of the present disclosure can reduce BM pooling to 2% wt/wt, as compared to TEXTOR brand nonwovens at 40% wt/wt. BM pooling values can be considered analogous to rewet values and represent BM on skin.

In a first particular aspect, a high topography nonwoven substrate includes synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, and wherein the density of a projection element is the same as the density of the base layer.

A second particular aspect includes the first particular aspect, wherein the binder fibers are bi- and/or multi-component binder fibers.

A third particular aspect includes the first and/or second aspect, wherein the shape of a cross-section of a projection element at the proximal end of the projection element is the same as the shape of a cross-section of a projection element at the distal end of the projection element.

A fourth particular aspect includes one or more of aspects 1-3, wherein the shape of a cross-section of a projection element at the proximal end of the projection element is different from the shape of a cross-section of a projection element at the distal end of the projection element.

A fifth particular aspect includes one or more of aspects 1-4, wherein the shape of a cross-section of a projection element is circular, oval, rectangular, or square.

A sixth particular aspect includes one or more of aspects 1-5, wherein the density of a projection element at the proximal end of the projection element is the same as the density of a projection element at the distal end of the projection element.

A seventh particular aspect includes one or more of aspects 1-6, wherein the basis weight of a projection element at the proximal end of the projection element is the same as the density a projection element at the distal end of the projection element.

An eighth particular aspect includes one or more of aspects 1-7, wherein the size of a cross-section of a projection element at the proximal end of the projection element is different from the size of a cross-section of a projection element at the distal end of the projection element.

A ninth particular aspect includes one or more of aspects 1-8, wherein each projection element has a uniform density.

A tenth particular aspect includes one or more of aspects 1-9, wherein the height of a projection element is greater than the width or diameter of that projection element.

An eleventh particular aspect includes one or more of aspects 1-10, further comprising cellulosic fibers.

A twelfth particular aspect includes one or more of aspects 1-11, wherein the substrate has a compression resistance that provides 20 cubic centimeters or more of void volume per gram of substrate at 0.6 kPa pressure.

A thirteenth particular aspect includes one or more of aspects 1-12, wherein the ratio of the height of a projection element to the width or diameter of a projection element is greater than 0.5.

A fourteenth particular aspect includes one or more of aspects 1-13, wherein the height of a projection element is greater than 3 mm.

A fifteenth particular aspect includes one or more of aspects 1-14, wherein the sidewalls have greater than 50 percent of fibers oriented in the Z-direction.

A sixteenth particular aspect includes one or more of aspects 1-15, wherein the synthetic binder fibers have an average length greater than 3 mm.

A seventeenth particular aspect includes one or more of aspects 1-16, wherein the projection elements have a density between 0.001 and 0.02 g/cc.

An eighteenth particular aspect includes one or more of aspects 1-17, wherein the projection elements are uniformly distributed in both the X- and Y-directions.

In a nineteenth particular aspect, a high topography nonwoven substrate includes synthetic binder fibers, wherein the fibers of the substrate are entirely synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, wherein the shape of a cross-section of a projection element at the proximal end of the projection element is the same as the shape of a cross-section of a projection element at the distal end of the projection element, and wherein the density of a projection element is the same as the density of the base layer.

In a twentieth particular aspect, a high topography nonwoven substrate includes synthetic binder fibers; a planar base layer having an X-Y surface and a backside surface opposite the X-Y surface; and a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, wherein each projection element has a uniform density, wherein the height of a projection element is greater than the width or diameter of that projection element, and wherein the density of a projection element is the same as the density of the base layer.

These and other modifications and variations to the present disclosure may be practiced by those of ordinary skill in the art, without departing from the spirit and scope of the present disclosure, which is more particularly set forth in the appended claims. In addition, it should be understood that aspects of the various aspects may be interchanged both in whole and in part. Furthermore, those of ordinary skill in the art will appreciate that the foregoing description is by way of example only, and is not intended to limit the disclosure so further described in such appended claims.

1. A high topography nonwoven substrate comprising:
synthetic binder fibers;
a planar base layer having an X-Y surface and a backside surface opposite the X-Y surface, the base layer having a density; and
a plurality of projection elements integral with and protruding in a Z-direction from the X-Y surface, wherein each projection element has a height, a diameter or width, a cross-section, a sidewall, a density, a proximal end where the projection element meets the base layer, and a distal end opposite the proximal end, wherein the projection elements are distributed in both the X- and Y-directions, and wherein the density of a projection element is the same as the density of the base layer.

2. The high topography nonwoven substrate of claim 1, wherein the binder fibers are bi- and/or multi-component binder fibers.

3. The high topography nonwoven substrate of claim 1, wherein a shape of a cross-section of a projection element at the proximal end of the projection element is the same as a shape of a cross-section of the projection element at the distal end of the projection element.

4. The high topography nonwoven substrate of claim 1, wherein a shape of a cross-section of a projection element at the proximal end of the projection element is different from a shape of a cross-section of the projection element at the distal end of the projection element.

5. The high topography nonwoven substrate of claim 1, wherein a shape of a cross-section of a projection element is circular, oval, rectangular, or square.

6. The high topography nonwoven substrate of claim 1, wherein a density of a projection element at the proximal end of the projection element is the same as a density of a the projection element at the distal end of the projection element.

7. The high topography nonwoven substrate of claim 1, wherein a basis weight of a projection element at the proximal end of the projection element is the same as a basis weight of the projection element at the distal end of the projection element.

8. The high topography nonwoven substrate of claim 1, wherein a size of a cross-section of a projection element at the proximal end of the projection element is different from a size of a cross-section of the projection element at the distal end of the projection element.

9. The high topography nonwoven substrate of claim 1, wherein each projection element has a uniform density.

10. The high topography nonwoven substrate of claim 1, wherein the height of a projection element is greater than the width or diameter of that projection element.

11. The high topography nonwoven substrate of claim 1, further comprising cellulosic fibers.

12. The high topography nonwoven substrate of claim 1, wherein the substrate has a compression resistance that provides 20 cubic centimeters or more of void volume per gram of substrate at 0.6 kPa pressure.

13. The high topography nonwoven substrate of claim 1, wherein a ratio of the height of a projection element to the width or diameter of the projection element is greater than 0.5.

14. The high topography nonwoven substrate of claim 1, wherein the height of a projection element is greater than 3 mm.

15. The high topography nonwoven substrate of claim 1, wherein the sidewalls have greater than 50 percent of fibers oriented in the Z-direction.

16. The high topography nonwoven substrate of claim 1, wherein the synthetic binder fibers have an average length greater than 3 mm.

17. The high topography nonwoven substrate of claim 1, wherein the projection elements have a density between 0.001 and 0.02 g/cc.

18. The high topography nonwoven substrate of claim 1, wherein the projection elements are uniformly distributed in both the X- and Y-directions.

19. A high topography nonwoven substrate comprising:
synthetic binder fibers, wherein the fibers of the substrate are entirely synthetic binder fibers;
a planar base layer having an X-Y surface and a backside surface opposite the X-Y surface, the base layer having a density; and
a plurality of projection elements integral with and protruding in a Z-direction from the X-Y surface, wherein each projection element has a height, a diameter or width, a cross-section, a sidewall, a density, a proximal end where the projection element meets the base layer, and a distal end opposite the proximal end, wherein the projection elements are distributed in both the X- and Y-directions, wherein a shape of a cross-section of a projection element at the proximal end of the projection element is the same as a shape of a cross-section of the projection element at the distal end of the projection element, and wherein the density of the projection element is the same as the density of the base layer.

20. A high topography nonwoven substrate comprising:
synthetic binder fibers;
a planar base layer having an X-Y surface and a backside surface opposite the X-Y surface, the base layer having a density; and
a plurality of projection elements integral with and protruding in a Z-direction from the X-Y surface, wherein each projection element has a height, a diameter or width, a cross-section, a sidewall, a proximal end where the projection element meets the base layer, and a distal end opposite the proximal end, wherein the projection elements are distributed in both the X- and Y-directions, wherein each projection element has a uniform density, wherein the height of a projection element is greater than the width or diameter of that projection element, and wherein the density of the projection element is the same as the density of the base layer.
